(19) United States
(12) Patent Application Publication
(10) Pub. No.: US 2023/0114972 A1
(43) Pub. Date: Apr. 13, 2023

(54) SNARE FOR REMOVAL OF IMPLANTED CARDIAC LEADS

(71) Applicant: PHILIPS IMAGE GUIDED THERAPY CORPORATION, SAN DIEGO, CA (US)

(72) Inventors: Tyler BIGHAM, BOULDER, CO (US); Michael BROWN, BOULDER, CO (US); Nathan WHITE, BOULDER, CO (US); David SCHECHTER, BOULDER, CO (US); Griffin PAUL, BOULDER, CO (US)

(21) Appl. No.: 17/910,784

(22) PCT Filed: Mar. 4, 2021
(86) PCT No.: PCT/EP2021/055407

§ 371 Date: Sep. 9, 2022

Related U.S. Application Data
(60) Provisional application No. 62/987,903, filed on Mar. 11, 2020.

(51) Int. Cl. A61B 17/22; A61B 17/00

(52) U.S. Cl. CPC A61B 17/22031; A61B 17/00234; A61B 2017/22035; A61B 2017/00358

(57) ABSTRACT

The present disclosure relates generally to medical devices and the use of medical devices for the treatment of vascular conditions. Particularly, the present disclosure provides devices and methods for using a snaring system to remove an implanted lead from the vasculature. A snaring system comprises an outer sheath (710) having a lumen; an inner sheath (705); a pusher (745) disposed within the lumen of the sheath, wherein the pusher comprises a longitudinal axis; a closed loop (720) having an opening, wherein the closed loop comprises a proximal extended portion, an S-curve portion and a distal extended portion, wherein the proximal extended portion of the closed loop is coupled to the pusher, wherein the proximal extended portion is substantially parallel to the longitudinal axis to the pusher, wherein the pusher and the closed loop are slidable relative to the inner sheath, whereupon being in a deployed configuration, the closed loop is disposed distally of the sheath and the S-curve portion is disposed substantially perpendicular to the proximal extended portion, wherein the S-curve portion comprises a first radius and a second radius, wherein the first radius is closer to the proximal extended portion relative to the distal extended portion, wherein the second radius is closer to the distal extended portion relative to the proximal extended portion, wherein the first radius is greater than the second radius; and a threader (725) fixedly coupled to the inner sheath, whereupon the closed loop being pulled proximally, the closed loop collapses over the threader and the threader is disposed within the opening of the closed loop. In one embodiment, two opposing closed loops are provided.

### FIELD

The present disclosure relates generally to medical devices and the use of medical devices for the retrieval and/or removal of implanted objects such as cardiac leads. In particular, the present disclosure provides devices and methods for using a snare and/or snare system to remove one or more implanted cardiac leads.

### BACKGROUND

Cardiac pacing systems, such as a pacemaker or a defibrillator, typically include one or more leads, which are placed inside the body of a patient. For example, the pacemaker includes a power source and circuitry configured to send timed electrical pulses to the lead. The lead carries the electrical pulse to the heart to initiate a cardiac cycle, and transmits information about the heart's electrical activity to the pacemaker. In some cases, a lead is inserted through a vein or artery (collectively vasculature) and guided to the heart where it is attached. In other instances, a lead is attached to the outside of the heart. At times, leads may be removed from patients for numerous reasons, including but not limited to, infections, lead age, and lead malfunction.

Current commercially available snares may not reliably capture the lead, thereby potentially leading to an increase in the overall time to complete the lead removal procedure as well as potentially elevating risk to the patient. Additionally, because the imaging equipment used to perform a lead removal procedure typically portrays the lead and snare in a two-dimensional portrayal, it may be difficult for the surgeon or clinician to visualize the three-dimensional aspects of the lead and snare during the procedure. As such, capturing a lead with a snare can be unpredictable.

### SUMMARY

What is needed is a more reliable snare that increases the likelihood of capturing the lead so the use of the snare is more predictable. Versions of the snare of the present disclosure also decrease the overall time to complete the lead removal procedure because the surgeon or clinician can more easily visualize the three-dimensional aspects of the lead relative to the snare. For example, the present disclosure discusses a snare that has a specially designed closed loop or leaflet that increases the likelihood that a clinician will be able to quickly and efficiently pass a threader through an opening in the closed loop or have the closed loop collapse over the threader, thereby creating a pinch point between the closed loop and the threader to secure the implanted object or lead. These and other needs are addressed by the various aspects, embodiments, and configurations of the present disclosure.

An example of a snaring system or snare for capturing a lead comprises: a sheath having a lumen; a pusher disposed within the lumen of the sheath, wherein the pusher comprises a longitudinal axis; a closed loop having an opening, wherein the closed loop comprises a proximal extended portion, an S-curve portion and a distal extended portion, wherein the proximal extended portion of the closed loop is coupled to the pusher, wherein the proximal extended portion is substantially parallel to the longitudinal axis to the pusher, wherein the pusher and the closed loop are slidable relative to the inner sheath, whereupon being in a deployed configuration, the closed loop is disposed distally of the sheath and the S-curve portion is disposed substantially perpendicular to the proximal extended portion, wherein the S-curve portion comprises a first radius and a second radius, wherein the first radius is closer to the proximal extended portion in relationship to or relative to the distal extended portion, wherein the second radius is closer to the distal extended portion in relationship to or relative to the proximal extended portion, wherein the first radius is greater than the second radius, and wherein the second radius is smaller than the first radius; and a threader fixedly coupled to the inner sheath, whereupon the closed loop being pulled proximally, the closed loop collapses over the threader and the threader is disposed within the opening of the closed loop.

Another example includes the snaring system of the previous paragraph, wherein the first radius is between about 0.15 inches and 0.50 inches, and wherein second radius is between about 0.10 inches and 0.25 inches.

Another example includes the snaring system of any of the previous paragraphs, wherein the first radius faces proximally.

Another example includes the snaring system of any of the previous paragraphs, wherein the second radius faces distally.

Another example includes the snaring system of any of the previous paragraphs, wherein the distal extended portion is disposed substantially perpendicular to the S-curve portion.

Another example includes the snaring system of any of the previous paragraphs, wherein the distal extended portion comprises a radius.

Another example includes the snaring system of any of the previous paragraphs, wherein the radius of the distal extended portion faces the threader.

Another example includes the snaring system of any of the previous paragraphs, wherein threader is substantially parallel to the longitudinal axis.

Another example includes the snaring system of any of the previous paragraphs, wherein the threader has a radius, and the radius of the threader faces the closed loop.

Another example includes the snaring system of any of the previous paragraphs, wherein a height from a beginning of the first radius to a distal end of the closed loop is about 0.80 inches and wherein a width from the beginning of the first radius to the distal end of the closed loop is about 0.60 inches

Another example includes the snaring system of any of the previous paragraphs, wherein the closed loop is constructed of a shape-memory metal.

A separate example of a snaring system or snare for capturing a lead comprises: a sheath having a lumen; a pusher disposed within the lumen of the sheath, wherein the pusher comprises a longitudinal axis; a first closed loop and a second closed loop, wherein each of the first closed loop and the second closed loop have an opening, wherein each of the first closed loop and the second closed loop comprises a proximal extended portion, an S-curve portion and a distal extended portion, wherein the respective proximal extended portions of the first closed loop and the second closed loops are coupled to the pusher, wherein the respective proximal extended portions of the first closed loop and the second closed loops are substantially parallel to the longitudinal axis of the pusher, wherein the pusher and the closed loop are slidable relative to the inner sheath, whereupon being in a deployed configuration, the first closed loop and the second closed loop are disposed distally of the sheath and the S-curve portions of the first closed loop and the second closed loop are disposed substantially perpendicular to respective proximal extended portions of the first closed loop and the second closed loop, wherein the S-curve portion of the first closed loop and the S-curve portion of the second closed loop extend in radially opposite directions; and a threader coupled to the inner sheath, whereupon the first closed loop and the second closed loop being pulled proximally, the second closed loop collapses over the first closed loop and the threader, the first closed loop collapses over the threader, and the threader is disposed within the respective openings of the first closed loop and the second closed loop.

Another example includes the snaring system of the previous paragraph, wherein the S-curve portion of the second closed loop is axially offset from the S-curve portion of the first closed loop.

Another example includes the snaring system of any of the previous paragraphs, wherein the proximal extended portion of the second closed loop is longer than the proximal extended portion of the first closed loop.

Another example includes the snaring system of any of the previous paragraphs, wherein each of the S-curve portions comprises a first radius and a second radius, wherein the first radius faces proximally and the second radius faces distally.

Another example includes the snaring system of any of the previous paragraphs, wherein each of the distal extended portions is disposed substantially perpendicular to each of the respective S-curve portions.

As used herein, “at least one,” “one or more,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B and C together. When each one of A, B, and C in the above expressions refers to an element, such as X, Y, and Z, or class of elements, such as X₁-Xₘ Y₁-Yₘ, and Z₁-Zₒ, the phrase is intended to refer to a single element selected from X, Y, and Z, a combination of elements selected from the same class (for example, X₁ and X₂) as well as a combination of elements selected from two or more classes (for example, Y₁ and Zₒ).

It is to be noted that the term “a” or “an” entity refers to one or more of that entity. As such, the terms “a” (or “an”), “one or more” and “at least one” can be used interchangeably herein. It is also to be noted that the terms “comprising,” “including,” and “having” can be used interchangeably.

The term “about” when used in conjunction with a numeric value shall mean plus and/or minus ten percent (10%) of that numeric value, unless otherwise specifically mentioned herein.

The term “catheter” as used herein generally refers to a tube that can be inserted into a body cavity, duct, lumen, or vessel, such as the vasculature system. In most uses, a catheter is a relatively thin, flexible tube (“soft” catheter), though in some uses, it may be a larger, solid, less flexible—but possibly still flexible—catheter (“hard” catheter). In some uses a catheter may contain a lumen along part or all of its length to allow the introduction of other catheters or guidewires.

The term “means” as used herein shall be given its broadest possible interpretation in accordance with 35 U.S.C. § 112(f). Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

The term “sheath” or “shaft” as used herein generally refers to a tube that can be inserted into a body cavity duct, lumen, or vessel, such as the vasculature system that allows for the introduction of other devices, such as catheters, and the introduction of fluid along its length. The sheath can have a closed end or an open end. Because the sheath is a tube that can be inserted into a body cavity, duct, lumen, or vessel, such as the vasculature system, the sheath and shaft may also be considered a catheter.

The terms “vasculature” and “vascular” as used herein refer to any part of the circulatory system of a subject, including peripheral and non-peripheral arteries and veins. Vasculature can be comprised of materials such as nucleic acids, amino acids, carbohydrates, polysaccharides, lipids fibrous tissue, calcium deposits, remnants of dead cells, cellular debris and the like.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein. Every number, including an integer (a whole number) or non-integer (a number that has fractional parts or a number with a decimal) between the numerical range given throughout this disclosure is deemed to include each and every integer and non-integer that falls within such numerical range, as if such integers and non-integers were all expressly written herein.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

### BRIEF DESCRIPTION OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1A is a perspective view an exemplary snaring system, including an inner shaft and an outer sheath, wherein a closed loop and a threader are coupled to a distal end of the inner shaft.

FIG. 1B is a side view of the exemplary snaring system illustrated in FIG. 1A.

FIG. 2A is a side view of the closed loop illustrated in FIGS. 1A and 1B.

FIG. 2B is a front view of the closed loop illustrated in FIGS. 2A.

FIG. 3A is a side view of the threader illustrated in FIGS. 1A and 1B.

FIG. 3B is a front view of the threader illustrated in FIGS. 3A.

FIG. 4A is an illustration of the snaring system depicted in FIGS. 1-3 with the closed loop in a fully expanded state adjacent a lead.

FIG. 4B is an illustration of the snaring system with the closed loop overlapping the lead and threader due to the pulling of the closed loop, wherein the closed loop in a partially collapsed state.

FIG. 4C is an illustration of the snaring system with the closed loop being further pulled into the outer sheath.

FIG. 4D is an illustration of the snaring system with the outer sheath being advanced distally over the closed loop and the threader.

FIG. 4E is an illustration of the snaring system with the outer sheath being further advanced distally over the closed loop and the threader such that both the closed loop and the threader, as well as a portion of the lead are disposed within the outer sheath.

FIG. 5A is a perspective view another exemplary snaring system, including an inner shaft and an outer sheath, wherein two opposing closed loops and a threader are coupled to a distal end of the inner shaft.

FIG. 5B is a side view of the other exemplary snaring system illustrated in FIG. 5A.

FIG. 6A is an illustration of the snaring system depicted in FIGS. 5A & 5B with the two opposing closed loops in a fully expanded state adjacent a lead.

FIG. 6B is an illustration of the snaring system with one closed loop overlapping the lead and another closed loop due to the pulling of the closed loops or the advancement of the outer sheath, wherein the closed loops are in a partially collapsed state.

FIG. 6C is an illustration of the snaring system with one closed loop overlapping the lead and another closed loop due to the additional pulling of the closed loops or advancement of the sheath, and the threader is disposed between the openings of the two closed loops.

FIG. 7A is a cut-away side view another exemplary snaring system.

FIG. 7B is a side view of the exemplary snaring system illustrated in FIG. 7A rotated 90 degrees about the longitudinal axes of the inner sheath and the outer sheath.

### DETAILED DESCRIPTION

The present disclosure relates generally to medical devices and the use of medical devices for the retrieval and/or removal of implanted objects such as cardiac leads. In particular, the present disclosure provides devices and methods for using a snare and/or snare system to remove the cardiac leads.

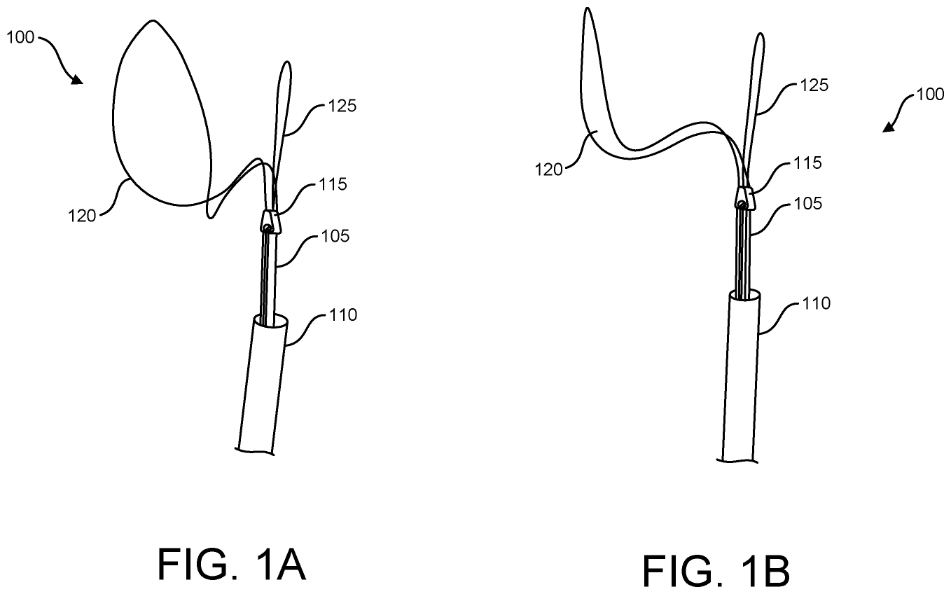

Referring to FIGS. 1A and 1B, there is depicted an exemplary snaring system 100 having an inner shaft 105 and an outer sheath 110, wherein a closed loop 120 and a threader 125 are both coupled to a distal end 115 of the inner shaft 105 in a fully deployed configuration. The closed loop 120 and the threader 125 may both be fixedly coupled to the distal end 115 of the inner shaft 105 or the closed loop 120 and the threader 125 may both be slidably coupled to the distal end 115 of the inner shaft 105, but it may be preferable for the closed loop 120 to be slidably coupled to the distal end 115 of the inner shaft 105 and the threader 125 to be fixedly coupled to the distal end 115 of the inner shaft 105, or vice versa, so that the closed loop 120 can slide relative to both the threader 125 and the distal end 115 of the inner shaft 105. As shown in FIGS. 1A and 1B, when the exemplary snaring system 100 is in a fully deployed configuration, the overall vertical length of the closed loop 120 is longer than the overall vertical length of the threader 125 in relation to the distal end 115 of the inner shaft 105 and the distal end of the outer sheath 110.

Both the closed loop 120 and the threader 125 are constructed of biocompatible metal, such as stainless steel or a shape memory alloy (e.g., nitinol). The closed loop 125 can also be made of a stainless-steel material as it does not need to have the same shape memory characteristics as closed loop 120. The diameter of the wire used to construct the closed loop 120 may have a diameter in the range between about 0.010 inches (0.254 millimeters) and about 0.020 inches (0.508 millimeters) including about 0.015 inches (0.381 millimeters).

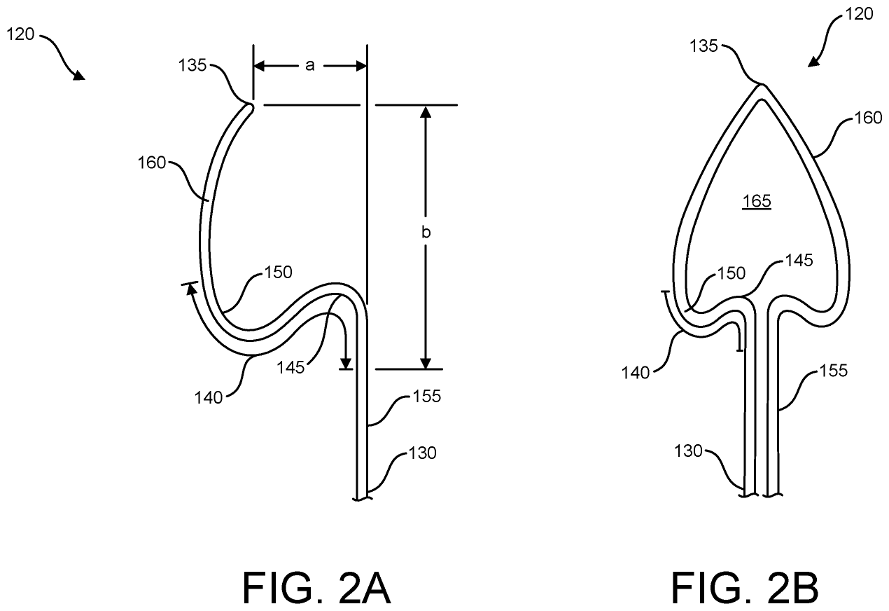

Referring to FIGS. 2A and 2B, there is depicted a closed loop 120 that is configured to have a predetermined shape, as discussed in more detail below, when the closed loop 120 is disposed beyond the distal end of the outer sheath 110 which is not shown in FIGS. 2A and 2B. The closed loop 120 has a proximal end 130 and a distal end 135. Referring to FIG. 2B, which is a front view of the closed loop 120, there are two sides to the closed loop, wherein each side is the mirror image of the other side extending from the proximal end 130 to the distal end 135. That is, the sides of the closed loop 120 are symmetrical to one another. As such, one side (i.e., the left side) of the closed loop 120 begins at the proximal end 130 and extends to the distal end 135, and because the closed loop 120 is constructed of a single continuous wire, the other side (i.e., the right side) of the closed loop 120 begins at the distal end 135 and extends to another proximal end 130. In other words, the closed loop 120 includes two sides having a common distal end 135 and two separate proximal ends 130—a separate proximal end 130 for each side.

Although FIGS. 2A and 2B depict the sides of the closed loop 120 as being symmetrically shaped, the present disclosure contemplates the two sides being asymmetrical relative to one another. For example, asymmetrically shaped sides of the closed loop 120 may provide certain clinical benefits, such as identifying the orientation of the closed loop 120 under fluoroscopy. For example, if the left side is straighter than the right side or the right side has a greater curve than the left side, then it may assist the physician in identifying whether the closed loop 120 is facing a particular direction, such as towards or away from a given reference point.

The two sides of the closed loop 120 are separated from one another and create an opening 165 in the closed loop 120. The greatest distance between the two sides is in the range between about 0.25 inches (6.35 millimeters) and about 1.0 inches (25.4 millimeters) including about 0.55 inches (14.0 millimeters). The distal end 135 of the closed loop 120 is curved. The radius of curvature of the distal end 135 is in the range between about 0.02 inches (0.508 millimeters) and about 0.60 inches (15.0 millimeters) including about 0.04 inches (1.0 millimeter). As shown in FIG. 2B, due to these dimensions, the face of the closed loop 120 has a petal or leaf configuration with an opening 165 therebetween.

Referring to FIG. 2A, each side of the closed loop 120 between the proximal end 130 and the distal end 135 are a proximal extended portion 155, an S-curve portion 140 and a distal extended portion 160. Upon viewing the closed loop 120 from the side, the proximal extended portion 155 is substantially straight and extends from the proximal end 130 to the S-curve portion 140. Continuing to view the closed loop 120 from the side, the S-curve portion 140 is disposed between the proximal extended portion 155 and the distal extended portion 160. The distal extended portion 155 may also be substantially straight and extends from the S-curve portion 140 to the distal end 135 of the closed loop 120. The S-curve portion 140 is, therefore, generally perpendicular to the proximal extended portion 155 and the distal extended portion 160, wherein the proximal extended portion 155 and the distal extended portion 160 are generally parallel to the longitudinal axis of the inner shaft 105.

The S-curve portion 140 has a first/proximal curve 145 and a second/distal curve 150. The first/proximal curve 145 is disposed relatively closer to the proximal extended portion 155 in relation to the distal extended portion 160. The second/distal curve 150 is disposed relatively closer to the distal extended portion 160 in relation to the proximal extended portion 155. It may be preferable for the first/proximal curve 145 to have a radius of curvature that is greater than the radius of curvature for the second/distal curve 150. In other words, it may be preferable for the radius of curvature for the second/distal curve 150 to be smaller than the radius of curvature for the first/proximal curve 145. For example, the first/proximal curve 145 may have a radius of curvature in the range between about 0.15 inches (3.81 millimeters) and about 0.75 inches (19.05 millimeters) including about 0.24 inches (6.1 millimeters), wherein the curve 145 faces the proximal end 130 of the closed loop 120. The second/distal curve 150 may have a radius of curvature in the range between about 0.075 inches (1.91 millimeters) and about 0.45 inches (11.43 millimeters) including about 0.16 inches (4.1 millimeters), wherein the curve 150 faces the distal end 135 of the closed loop 120. the first radius is greater than the second radius, and wherein the second radius is smaller than the first radius

Continuing to view the closed loop 120 from the side, the distal extended portion 160 is substantially straight and extends from the S-curve portion 140 to the distal end 135. The distal extended portion 160 has a radius of curvature in the range between about 0.4 inches (10.16 millimeters) and no curvature, including about 0.98 inches (24.9 millimeters), wherein the curve of the distal extended portion 160 faces a direction generally perpendicular to the longitudinal axis of the proximal extended portion 155 of the closed loop 120.

Continuing to refer to FIG. 2A, which is a side view the closed loop 120, the axial length (b) between the beginning of the S-curve portion 140 (or the end of the proximal extended portion 155) to the distal end 135 (of the distal extended portion 160) is in the range between about 0.70 inches (17.78 millimeters) and about 1.40 inches (35.56 millimeters) including about 0.80 inches (20.3 millimeters). The radial length (a) between the beginning of the S-curve portion 140 (or the end of the proximal extended portion 155) to the distal end 135 is in the range between about 0.50 inches (12.70 millimeters) and about 1.25 inches (31.75 millimeters) including about 0.60 inches (15.2 millimeters). Viewed from its side, the closed loop 120 appears to have a “field goal” shape. As mentioned above, viewed from its face, the closed loop 120 appears to have a petal or leaf configuration. So, from a perspective view, the closed loop 120 appears to have a “field goal petal” shape.

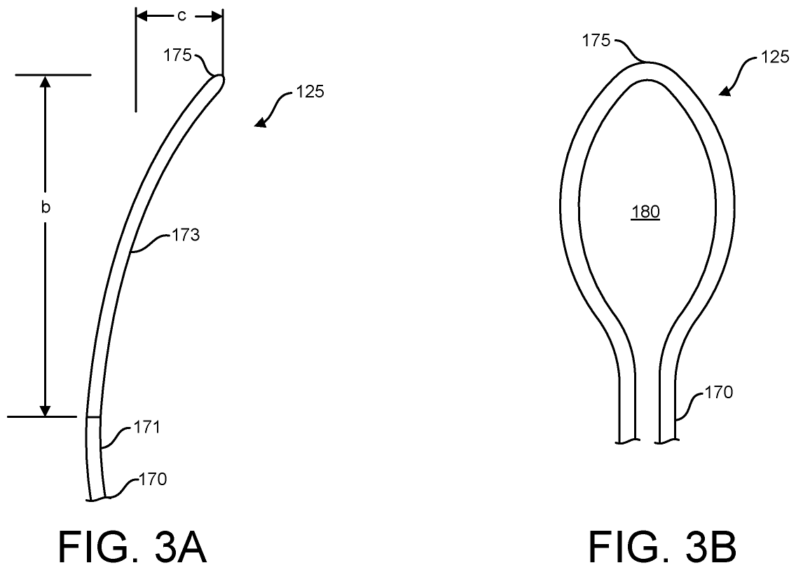

Referring to FIGS. 3A and 3B, there is depicted a threader 125 that is configured to have a predetermined shape, as discussed in more detail below, when the threader 125 is disposed beyond the distal end of the outer sheath 110. The threader 125 has a proximal end 170 and a distal end 175. Referring to FIG. 3B, which is a front view of the threader 125, there are two sides to the threader 125, wherein each side is the mirror image of the other side extending from the proximal end 170 to the distal end 175. That is, the sides of the threader 125 are symmetrical to one another. As such, one side (i.e., the left side) of the threader 125 begins at the proximal end 170 and extends to the distal end 175, and because the threader 125 is constructed of a single continuous wire, the other side (i.e., the right side) of the threader 125 begins at the distal end 175 and extends to another proximal end 170. In other words, the threader 125 includes two sides having a common distal end 175 and two separate proximal ends 170—a separate proximal end 170 for each side. The two sides are separated from one another and create an opening 180 in the threader 125. The greatest distance between the two sides is in the range between about 0 inches (0 millimeters) and about 0.35 inches (8.89 millimeters) including about 0.057 inches (1.45 millimeters). The distal end 175 of the threader 125 is curved. The radius of curvature of the distal end 175 is in the range between about 0.020 inches (0.508 millimeters) and about 0.125 inches (3.175 millimeters) including about 0.042 inches (0.5 millimeter).

Although FIGS. 3A and 3B depict the sides of the threader 125 as being symmetrically shaped, the present disclosure contemplates the two sides being asymmetrical to one another. For example, asymmetrically shaped sides of the threader 125 may provide certain clinical benefits, such as identifying the orientation of the threader 125 under fluoroscopy. For example, if the left side is straighter than the right side or the right side has a greater curve than the left side, it may assist the physician in identifying whether the threader 125 is facing a particular direction, such as towards or away from a given reference point.

Referring to FIG. 3A, which is a side view of the threader 125, the threader 125 has a proximal extended portion 171 and a distal extended portion 173, wherein the proximal extended portion 171 is parallel to the longitudinal axis of the inner shaft 105. The proximal extended portion 171 is substantially straight, and the distal extended portion 173 has a radius of curvature in the range between about 0.50 inches (12.7 millimeters) and about no curve including about 0.69 inches (17.5 millimeters), wherein the curve faces toward or generally perpendicular to the axis of the proximal extended portion 171. Continuing to view the threader 125 from the side, the axial length (d) of the extended distal portion 173 is in the range between about 0.40 inches (10.16 millimeters) and about 2.0 inches (50.8 millimeters) including about 0.50 inches (12.7 millimeters). The radial length (c) of the extended distal portion 173 is in the range between about 0 inches (0 millimeters) and about 0.25 inches (6.35 millimeters) including about 0.17 inches (4.3 millimeters). Although the beginning of the radial length (c) of the extended distal portion 173 can be measured beginning at any point along the extended distal portion 173 to its distal end 175, it may be preferable for the radial length (c) of the extended distal portion 173 to be measured beginning at the distal end of the proximal extended portion 171 (the proximal end of the extended distal portion 173) and extending to the distal end 175 of the extended distal portion 173.

Although FIGS. 3A and 3B depict the threader 125 as having two sides that form a continuous, closed loop, the present disclosure envisions the threader as having only a single protruding member or wire without any loop. For example, the threader may be constructed of a single, round wire or a flat wire having a rectangular cross section. It may also be desirable for the threader having a single protruding member to include an atraumatic tip at its distal end, thereby ensuring blunt contact between the single wire and tissue or vasculature to prevent or minimize or reduce the potential risk of accidental puncture.

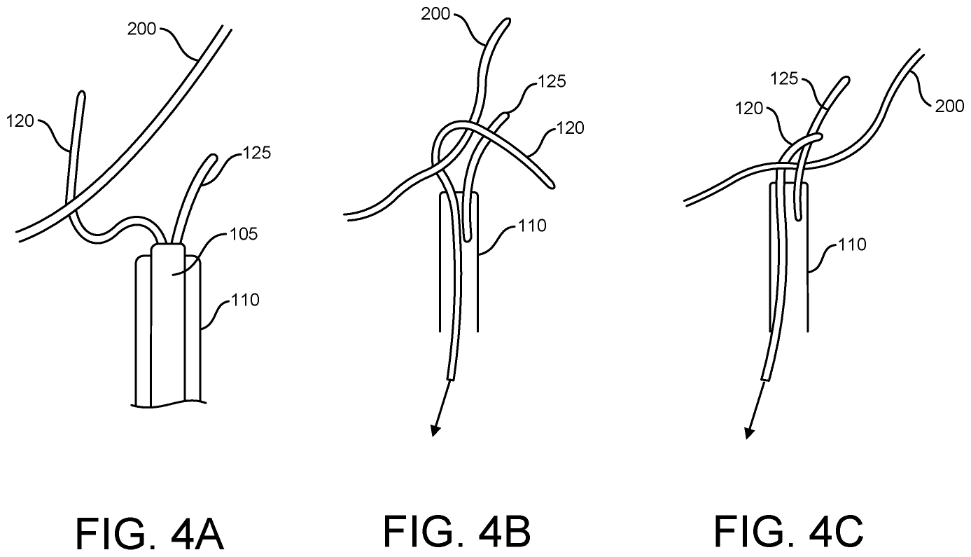

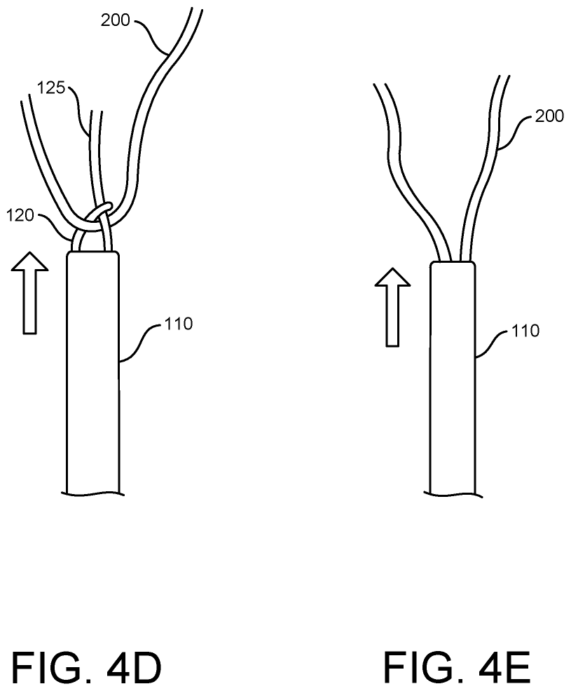

Referring to FIGS. 4A to 4E, there is depicted a series of illustrations demonstrating how a clinician can use the snaring system 100 of the present disclosure, as described above with respect to FIGS. 1-3. Referring to FIG. 4A, which illustrates the sharing system 100 in a fully deployed configuration, the inner shaft 105 extends to or beyond of the distal end of the outer sheath 110, thereby allowing the closed loop 120 and threader 125 to assume their respective predetermined shapes because neither the closed loop 120 nor the threader 125 are constrained by the outer sheath 110. When the closed loop 120 and threader 125 are in their predetermined shapes, the clinician places the closed loop 120 adjacent a lead 200 such that the lead 200 is disposed between the closed loop 120 and the threader 125. Referring to FIG. 4B, the clinician pulls the proximal end of the closed loop 120 in a proximal direction and/or pushes the outer sheath 110 in a distal direction, and the closed loop 120 begins to enter the lumen of the outer sheath 110. Due to the specifications of the closed loop 120 discussed above, such as the closed loop's wire material, wire diameter, dimensions and shapes of the different portions (i.e., proximal extended portion, S-curve portion and distal extended portion) of the closed loop 120, as the closed loop 120 enters the lumen of the outer sheath 110, the closed loop 120 begins to collapse and overlap the lead 200 and threader 125, wherein the threader 125 enters the opening 165 in the closed loop 120 because the open loop 165 collapses and passes over the threader 125. In other words, as the threader 125 remains stationary, the open loop 165 moves and collapses over the threader 125 such that the threader 125 enters the opening 165 in the closed loop 120. As such, the lead 200 is captured between both sides of the closed loop 120 and the threader 125.

Referring to FIG. 4C, the clinician continues to pull the closed loop 120 proximally into the outer sheath 110 until the exposed length of the closed loop 120 extending beyond the outer sheath 110 is shorter than the exposed length of the threader 125 extending beyond the outer sheath 110, thereby ensuring that the closed loop 120 and threader 125 have a solid grasp or “pinching action” on the lead 200. Referring to FIGS. 4D & 4E, the clinician pushes the outer sheath 110 distally until the combination of the closed loop 120, the threader 125 and the grasped portion of the lead 200 are disposed within the outer sheath 110, at which time the lead 200 may be removed from the subject's vasculature.

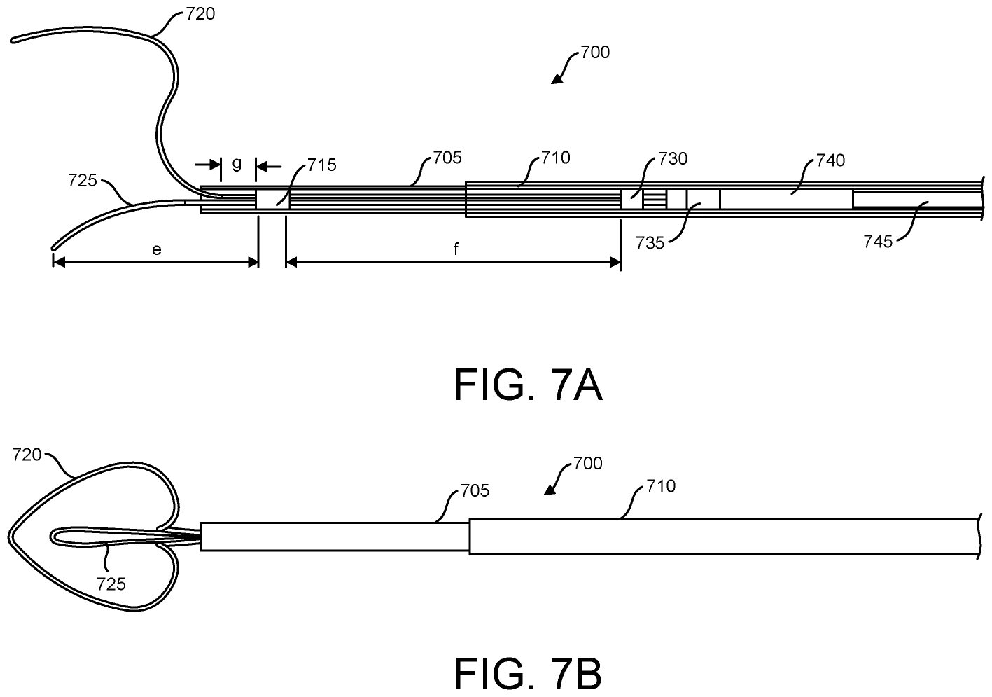

Referring to FIGS. 7A & 7B, there is depicted another exemplary snaring system 700. The snaring system 700 comprises an inner sheath 705 and an outer sheath 710, wherein the inner sheath 705 is slidably disposed within the outer sheath 710. The snaring system 700 also comprises a closed loop 720 and a threader 725. The closed loop 720 and the threader 725 shown in FIGS. 7A & 7B are the same as the closed loop 120 and threader 125 illustrated and discussed hereinabove with respect to FIGS. 1, 2 and 3.

Included within the inner sheath 705 are a distal collar 715, a middle collar 730, a proximal collar 735, an adapter collar 740 and a pusher 745. These items are listed in accordance with how they are arranged relative (e.g., distally or proximally) to another beginning with the item that is most distal and ending with the item that is most proximal. For example, the distal collar 715 is disposed distally of the middle collar 730, the proximal collar 735, the adapter collar 740 and the pusher 745, and the pusher 745 is disposed proximally of the distal collar 715, the middle collar 730, the proximal collar 735 and the adapter collar 740.

The wire that forms the closed loop 720 is fixedly coupled to the distal collar 715, and the distal collar 715 is fixedly coupled to the proximal collar 735, the adapter collar 740 or the pusher 745, and all of these items are fixedly coupled to one another such that these items translate axially along the longitudinal axis of the inner sheath 705 in unison. That is, if either of the closed loop 720, the distal collar 715, the proximal collar 735, the adapter collar 740 and the pusher 745 move axially, so do all of the other listed items. In other words, the closed loop's 720 axially movement is controlled by corresponding movement of the pusher 745.

The middle collar 730 is fixedly coupled to the inner sheath 705 within the lumen of the inner sheath 705. The proximal end(s) of the wire that forms the threader 725 are fixedly coupled to the middle collar 730. The wire that forms the closed loop 720 is slidably coupled to the middle collar 730 such that both ends of the wire pass through one or two lumens within the middle collar 730. Hence, the threader 725 is stationary relative to the inner sheath 705 and the middle collar 730. However, the closed loop 720, the distal collar 715, the proximal collar 735, the adapter collar 740 and the pusher 745 slide axially relative to the middle collar 730, the threader 725 and the inner sheath 705.

Again, the closed loop 720 is axially slidable relative to the inner sheath 705 along the inner sheath's 705 longitudinal axis, and the threader 725 is fixed relative to the inner sheath 705. As described above, the pusher 745 controls the movement of the closed loop 720 relative to the inner sheath 705. Referring to FIG. 7A & 7B, which depict the snaring system 700 in a fully deployed state or deployed configuration, a distal portion of the closed loop 720 is extending beyond the distal end of the inner sheath 705, and a proximal portion of the closed loop 720 is disposed within the lumen of the inner sheath 705. For example, as shown in FIG. 7A, the proximal extended portion of the closed loop 720 is entirely disposed within the inner sheath 705, and the proximal end of the S-curve portion of the closed loop 720 is disposed within the inner sheath 705 such that the length (g) of the proximal extended portion of the closed loop 720 is about 0.125 inches (0.32 millimeters) between the distal end of the distal collar 715 and the beginning of the S-curve portion of the closed loop 720. Also when the snaring system 700 in a deployed state or deployed configuration, the distal end of distal extended portion of the threader 725 is at a distance (e) relative to the distal end of the distal collar 715, wherein the distance (e) is about 0.75 inches (19.05 millimeters). Additionally, when the snaring system 700 is in a deployed state or deployed configuration, the length (f) of the wire of the closed loop 720 between the proximal end of the distal collar 715 and the distal end of the middle collar 730 is about 1.25 inches (31.75 millimeters). Moreover, when the snaring system 700 in a fully deployed state or deployed configuration, the distal end of the proximal collar 735 abuts the proximal end of the middle collar 730. And when the snaring system 700 in a fully collapsed or retracted state or fully collapsed or retracted configuration, the proximal end of the distal collar 715 abuts the distal end of the middle collar 730, and the closed loop 720 is completely or nearly completely disposed within the lumen of the inner sheath 705. Hence, the pusher 745 has a stroke of a distance (f) between the fully deployed state and the fully retracted state.

Using the snaring system 700 would be similar to the use of the snaring system 100 described above with respect to FIGS. 4A-4E except that the inner shaft 105, the outer sheath 110, the closed loop 120, and the threader 125 in FIGS. 1, 2 and 3 are replaced with the distal collar 715, middle collar 730, proximal collar 735, adapter collar 740, pusher 745, the inner sheath 705, the closed loop 720, and the threader 725 in FIGS. 7A and 7B.

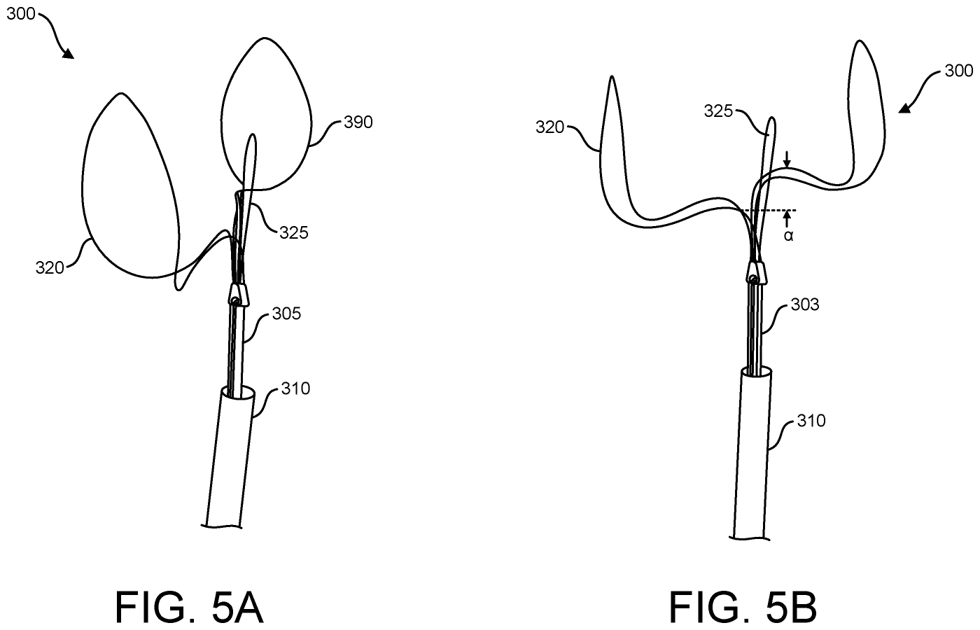

Referring to FIGS. 5A and 5B, there is depicted another exemplary snaring system 300 which may include an inner shaft 305, an outer sheath 310, two opposing closed loops 320, 390 and a threader 325, wherein the closed loops 320, 390 and the threader 325 are coupled to a distal end of the inner shaft 305. The closed loops 320, 390 and the threader 325 may both be fixedly coupled to the distal end of the inner shaft 305, but it may be preferable for the closed loops 320, 390 to be slidably coupled to the distal end of the inner shaft 305 and the threader 325 may to be fixedly coupled to the distal end of the inner shaft 305 or vice versa so that the closed loops 320, 390 can slide relative to the threader 325 and the distal end of the inner shaft 305. As shown in FIGS. 5A and 5B, when the exemplary snaring system 300 is in a fully deployed configuration, the vertical length of the closed loops 320, 390 are longer than the vertical length of the threader 325 in relation to the distal end of the inner shaft 305 and the distal end of the outer sheath 310.

The primary difference between the snaring system 300 illustrated in FIGS. 5A and 5B in comparison to the snaring system 100 illustrated in FIGS. 1A and 1B is that the snaring system 300 in FIGS. 5A and 5B has two opposing closed loops 320, 390, and the snaring system 100 in FIGS. 1A and 1B has one closed loop 120. The material, dimensions, configuration, etc. of the closed loop 320 and threader 325 of the snaring system 300 illustrated in FIGS. 5A and 5B are substantially similar to or the same as the closed loop 120 and threader 125 of the snaring system 100 illustrated in FIGS. 1A and 1B. The material, dimensions, configuration, etc. of the closed loop 390 of the snaring system 300 illustrated in FIGS. 5A and 5B is substantially similar to the closed loop 320 in FIGS. 5A and 5B (and substantially similar to the closed loop 120 of the snaring system 100 illustrated in FIGS. 1A and 1B) except that the closed loop 390 has a longer proximal extended portion than the proximal extended portion of closed loop 320 (and closed loop 120). Because the proximal extended portion of the closed loop 390 of the snaring system 300 is longer than that of the closed loop 320, the S-curve portions of the closed loops 320, 390 are axially offset from one another such that the S-curve portion of the closed loop 390 is disposed longitudinally distally of the S-curve portion the closed loop 320. In other words, when the snaring system 300 is in a fully deployed configuration, such as depicted in FIGS. 5A and 5B, the S-curve portion of the closed loop 320 is disposed longitudinally proximally of the S-curve portion the closed loop 390.

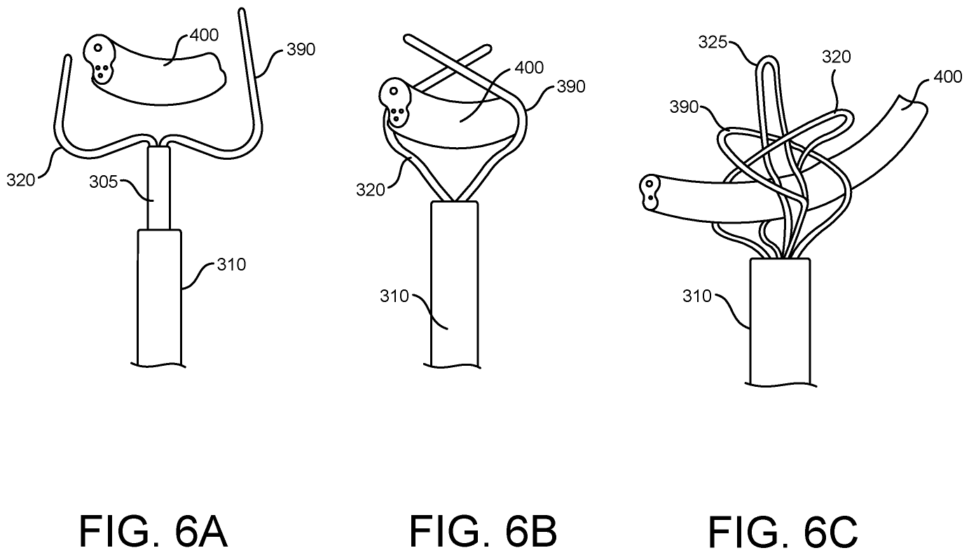

Because the proximal extended portion of the closed loop 390 of the snaring system 300 is longer than that of the closed loop 320 and the S-curve portions of the closed loops 320, 390 are axially offset from one another such that the S-curve portion of the closed loop 390 is disposed longitudinally distally of the S-curve portion the closed loop 320 in a fully deployed configuration, the closed loops 320, 390 and the threader 325 capture the lead 400 as illustrated in FIGS. 6A, 6B and 6C when a clinician pulls on the wires forming the closed loops 320, 390. Referring to FIG. 6A, the inner shaft 305 extends to or beyond of the distal end of the outer p sheath 310, thereby allowing the closed loops 320, 390 and threader 325 (not shown) to assume their respective predetermined shapes because neither the closed loops 320,390 nor the threader 325 are constrained by the outer sheath 310. When the closed loops 320, 390 and threader 325 (not shown) are in their predetermined shapes, the clinician places the closed loops 320, 390 adjacent a lead 400 such that the lead 400 is disposed between the closed loops 320, 390 and the threader 325 (not shown). Referring to FIG. 6B, the clinician pulls proximally on the proximal end of the closed loops 320, 390 and/or pushes distally on the outer sheath 310, and the inner sheath 305 and the closed loops 320, 390 begins to enter the lumen of the outer sheath 310. Due to the specifications of the closed loops 320, 390 discussed above, such as the closed loop's wire material, wire diameter, dimensions and shapes of the different portions of the closed loops 320, 390, as the closed loops 320, 390 enters the lumen of the outer sheath 310, the closed loops 320, 390 begin to collapse and overlap the lead 400 and threader 325, wherein the threader 325 enters the openings in the closed loops 320, 390.

Because the proximal extended portion of the closed loop 390 of the snaring system 300 is longer than that of the closed loop 320 and the S-curve portions of the closed loops 320, 390 are axially offset from one another such that the S-curve portion of the closed loop 390 is disposed longitudinally distally of the S-curve portion of the closed loop 320 in a fully deployed configuration, as the closed loops 320, 390 enter into the lumen of the outer sheath 310, the closed loop 320 begins to collapse prior to the closed loop 390 collapsing. Also, closed loop 320 begins to collapse prior to the closed loop 390 collapsing, closed loop 320 enters the opening of closed loop 390. As shown in FIG. 6C, as both of the closed loops 320,

390 collapse over the threader 325, the threader 325 enters the respective openings of the closed loops 320, 390, thereby capturing the lead 400. And as the clinician continues to pull the closed loops 320, 390 proximally into the outer sheath 310, the exposed lengths of the closed loops 320, 390 extending beyond the outer sheath 310 become shorter than the exposed length of the threader 325 extending beyond the outer sheath 325, thereby ensuring that the closed loops 320, 390 and threader 325 have a solid grasp of the lead 400. After having a solid grasp of the lead 400, the clinician pushes the outer sheath 310 further distally until the combination of the closed loops 320, the threader 325 and the grasped portion of the lead 300 are disposed within the outer sheath 310, at which time the lead 400 may be removed from the subject's vasculature.

It shall also be understood that this disclosure contemplates modifying the snaring system 700 illustrated in FIGS. 7A and 7B by adding a second closed loop opposite the first closed loop 720 to perform the procedure discussed hereinabove with respect to FIGS. 6A, 6B and 6C.

The present disclosure, in various aspects, embodiments, and configurations, includes components, methods, processes, systems and/or apparatus substantially as depicted and described herein, including various aspects, embodiments, configurations, sub combinations, and subsets thereof. Those of skill in the art will understand how to make and use the various aspects, aspects, embodiments, and configurations, after understanding the present disclosure. The present disclosure, in various aspects, embodiments, and configurations, includes providing devices and processes in the absence of items not depicted and/or described herein or in various aspects, embodiments, and configurations hereof, including in the absence of such items as may have been used in previous devices or processes, for example, for improving performance, achieving ease and\or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more, aspects, embodiments, and configurations for the purpose of streamlining the disclosure. The features of the aspects, embodiments, and configurations of the disclosure may be combined in alternate aspects, embodiments, and configurations other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed aspects, embodiments, and configurations. Thus, the following claims are hereby incorporated into this Detailed Description, with each claim standing on its own as a separate preferred embodiment of the disclosure.

Moreover, though the description of the disclosure has included description of one or more aspects, embodiments, or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and configurations to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A snaring system comprising:
a sheath having a lumen;
a pusher disposed within the lumen of the sheath, wherein the pusher comprises a longitudinal axis;
a closed loop having an opening, wherein the closed loop comprises a proximal extended portion, an S-curve portion and a distal extended portion, wherein the proximal extended portion of the closed loop is coupled to the pusher, wherein the proximal extended portion is substantially parallel to the longitudinal axis to the pusher, wherein the pusher and the closed loop are slidable relative to the inner sheath, whereupon being in a deployed configuration, the closed loop is disposed distally of the sheath and the S-curve portion is disposed substantially perpendicular to the proximal extended portion, wherein the S-curve portion comprises a first radius and a second radius, wherein the first radius is closer to the proximal extended portion relative to the distal extended portion, wherein the second radius is closer to the distal extended portion relative to the proximal extended portion, wherein the first radius is greater than the second radius; and
a threader fixedly coupled to the inner sheath, whereupon the closed loop being pulled proximally, the closed loop collapses over the threader and the threader is disposed within the opening of the closed loop.

2. The snaring system of claim 1, wherein the first radius faces proximally.

3. The snaring system of claim 3, wherein the second radius faces distally.

4. The snaring system of claim 1, wherein the distal extended portion is disposed substantially perpendicular to the S-curve portion.

5. The snaring system of claim 1, wherein the distal extended portion comprises a radius.

6. The snaring system of claim 5, wherein the radius of the distal extended portion faces the threader.

7. The snaring system of claim 1, wherein threader is substantially parallel to the longitudinal axis.

8. The snaring system of claim 7, wherein the threader has a radius, and the radius of the threader faces the closed loop.

9. The snaring system of claim 1, wherein a height from a beginning of the first radius to a distal end of the closed loop is about 0.80 inches and wherein a width from the beginning of the first radius to the distal end of the closed loop is about 0.60 inches

10. The snaring system of claim 1, wherein the closed loop is constructed of a shape-memory metal.

11. A snaring system comprising:
a sheath having a lumen;
a pusher disposed within the lumen of the sheath, wherein the pusher comprises a longitudinal axis;
a first closed loop and a second closed loop, wherein each of the first closed loop and the second closed loop have an opening, wherein each of the first closed loop and the second closed loop comprises a proximal extended portion, an S-curve portion and a distal extended portion, wherein the respective proximal extended portions of the first closed loop and the second closed loops are coupled to the pusher, wherein the respective proximal extended portions of the first closed loop and the second closed loops are substantially parallel to the longitudinal axis of the pusher, wherein the pusher and the closed loop are slidable relative to the inner sheath, whereupon being in a deployed configuration, the first closed loop and the second closed loop are disposed distally of the sheath and the S-curve portions of the first closed loop and the second closed loop are disposed substantially perpendicular to respective proximal extended portions of the first closed loop and the second closed loop, wherein the S-curve portion of the first closed loop and the S-curve portion of the second closed loop extend in radially opposite directions; and
a threader coupled to the inner sheath, whereupon the first closed loop and the second closed loop being pulled proximally, the second closed loop collapses over the first closed loop and the threader, the first closed loop collapses over the threader, and the threader is disposed within the respective openings of the first closed loop and the second closed loop.

12. The snaring system of claim 11, wherein the S-curve portion of the second closed loop is axially offset from the S-curve portion of the first closed loop.

13. The snaring system of claim 12, wherein the proximal extended portion of the second closed loop is longer than the proximal extended portion of the first closed loop.

14. The snaring system of claim 11, wherein each of the S-curve portions comprises a first radius and a second radius, wherein the first radius faces proximally and the second radius faces distally.

15. The snaring system of claim 11, wherein each of the distal extended portions is disposed substantially perpendicular to each of the respective S-curve portions.

16. A snaring system comprising:
a sheath having a lumen;
a pusher disposed within the lumen of the sheath, wherein the pusher comprises a longitudinal axis; and
a closed loop having an opening, wherein the closed loop comprises a proximal extended portion, an S-curve portion and a distal extended portion, wherein the proximal extended portion of the closed loop is coupled to the pusher, wherein the proximal extended portion is substantially parallel to the longitudinal axis to the pusher, wherein the pusher and the closed loop are slidable relative to the inner sheath, whereupon being in a deployed configuration, the closed loop is disposed distally of the sheath and the S-curve portion is disposed substantially perpendicular to the proximal extended portion, wherein the S-curve portion comprises a first radius and a second radius, wherein the first radius is closer to the proximal extended portion relative to the distal extended portion, wherein the second radius is closer to the distal extended portion relative to the proximal extended portion, wherein the first radius is greater than the second radius.
